(19) United States
(12) Patent Application Publication
(10) Pub. No.: US 2019/0282522 A1
(43) Pub. Date: Sep. 19, 2019

(54) ANESTHETIC COMPOSITION WITH REDUCED PAIN UPON INFILTRATION

(71) Applicant: UNIVERSITY OF CENTRAL FLORIDA RESEARCH FOUNDATION, INC., Orlando, FL (US)

(72) Inventors: David WEINSTEIN, Orlando, FL (US); Alex MOSES, St. Cloud, FL (US)

(21) Appl. No.: 16/281,628

(22) Filed: Feb. 21, 2019

Related U.S. Application Data
(60) Provisional application No. 62/633,388, filed on Feb. 21, 2018.

(51) Int. Cl. A61K 31/167; A61K 9/00; A61K 31/137; A61P 23/00

(52) U.S. Cl. CPC A61K 31/167; A61P 23/00; A61K 31/137; A61K 9/0019

(57) ABSTRACT

Disclosed herein are lidocaine compositions that are formulated to significantly reduce pain. Also disclosed are methods of using lidocaine compositions to induce an anesthetic effect in a patient. Articles of manufacture containing the lidocaine composition are also disclosed.

### BACKGROUND

Local anesthesia is administered in over one million cutaneous surgeries per year. Administration of local anesthesia is considered to be the most painful part of minor surgery. The most likely cause of pain is the low pH of the anesthetic solution. The obvious fix would be to increase pH. However, increasing pH causes degradation of epinephrine—a potent vasoconstrictor that acts to keep lidocaine localized. Premature degradation of epinephrine leads to diminished shelf life of the anesthetic solution. Benzyl alcohol—an alternative preservative—has shown potential at reducing pain, while not affecting pH of the solution. Additional factors that could be affecting pain include other components of the anesthetic solution such as lidocaine (anesthetic), epinephrine (vasoconstrictor), methylparaben (preservative), and saline (solvent). There is a lack of literature comparing of how all of these factors effect pain upon infiltration, Zaiac et al. J. Drugs in Dermatol, 2012; 11(10):e39-e42.

### SUMMARY

Disclosed embodiments are based on the discovery of the causative factors of pain in anesthetic solutions. Equipped with this information, new anesthetic formulations have been generated that are able to provide the requisite anesthetic effect and duration with a significantly lower induction of pain. Accordingly, one embodiment pertains to a composition comprising an aqueous solution of 0.5 to 0.05% w/v lidocaine in combination with epinephrine at approximately 1:1,000,000. In a more specific embodiment, the lidocaine percentage is or approximately is 0.1% w/v. Optionally, the improved anesthetic compositions may further comprise normal saline or bacteriostatic normal saline. In a specific example, the composition comprises a 1:9 solution of lidocaine (at approximately 1%) and epinephrine (at approximately 1:100,000) with bacteriostatic normal saline: composition of 0.1% w/v lidocaine with epinephrine 1:1,000,000 and bacteriostatic saline.

The disclosed anesthetic compositions may also include a buffering agent. In a specific example the buffering agent is sodium bicarbonate. The disclosed anesthetic compositions may also include benzyl alcohol.

Embodiments also pertain to an article of manufacture that include an amount of a disclosed anesthetic composition. Specific examples of articles of manufacture include vials or syringes.

Another embodiment includes a method of inducing an anesthetic effect in a patient comprising administering an effective amount an anesthetic composition of the present disclosure. Modes of administration include intradermal injection, subcutaneous injection, or intramuscular injection of the composition.

These and other embodiments are described further herein.

### BRIEF DESCRIPTION OF DRAWINGS

The accompanying drawings, which are incorporated herein and constitute part of this specification, illustrate exemplary embodiments of the disclosed subject matter, and, together with the general description given above and the detailed description given below, serve to explain the features of the disclosed subject matter.

FIG. 1 provides a diagram showing the pain scoring used in the experimental protocol.

FIG. 2 provides a chart showing the median duration of anesthetic solutions.

FIG. 3 provides a chart showing median pain of anesthetic solutions. Solution 1 (standard solution) was significantly more painful than solution 2, solution 7, and solution 8.

FIG. 4 provides a chart showing the effect of lidocaine dilution on pain. Solution 1 (1% lidocaine) was more significantly painful than solution 7 (0.1% lidocaine)*, but not more significantly more painful than solution 3 (0.5% lidocaine) or solution 5 (0.25% lidocaine).

*Solution 7 differed from solution 1 by two independent variables: concentration of lidocaine and the inclusion of benzyl alcohol (solution 7 had benzyl alcohol; solution 1 did not)

FIG. 5 provides a chart showing the effect of pH on pain with 1% lidocaine solution. Significant difference in pain between solution 1 and solution 2. The only independent variable changed between the two solutions was pH.

FIG. 6 provides a chart showing the effect of benzyl alcohol on pain. No significant difference in pain when comparing solutions with benzyl alcohol vs. without benzyl alcohol. Comparison was made at 0.5% lidocaine and at 0.25% lidocaine.

FIG. 7 provides a table 1 summarizing results of experimental protocol. No significant difference between duration of any of the 8 anesthetic solutions. Significant difference in pain when comparing solution 1 (standard solution) with solution 2, solution 7, and solution 8. *pH values were recorded after experiment for reference.

### DETAILED DESCRIPTION

The present disclosure is based on an evaluation of duration of anesthetic effect and pain infiltration on different anesthetic solutions. It has been discovered that a fundamental cause of pain upon infiltration is due to the anesthetic itself. However, until now, it was not known whether significant dilutions of the anesthetic would be sufficient to achieve an appropriate anesthetic effect and provide sufficient duration of effect. Based on the study disclosed herein, it has been found that a significant dilution of an anesthetic 0.1% can still provide an appropriate anesthetic or analgesic effect but with lower pain induction. Based on this discovery, provided herein are novel anesthetic solutions that reduce infiltration pain while providing clinically sufficient anesthetic effects.

According to one embodiment, disclosed is a composition that comprises 0.5 to 0.05% lidocaine in combination with epinephrine at approximately 1:1,000,000. In a specific embodiment, the lidocaine percentage is or approximately is 0.1%. The term “approximately” as used in reference to the concentration of lidocaine means a 25% decrease or increase in the stated value. For example, approximately 0.1% means a concentration ranging from 0.075% to 0.125%. In reference to a ratio, for example, the ratio of approximately 1:1,000,000 includes a ratio with the value ranges of 0.75-1.25:1,000,000.

In another example, the composition includes bacteriostatic normal saline and optionally buffered with a buffering compound such as sodium bicarbonate or NaH2PO4.

According to a further embodiment, provided is a method of inducing a localized anesthetic effect in a patient that involves injecting a composition comprising 0.5 to 0.05% lidocaine in combination with epinephrine approximately 1:1,000,000 into the patient. The injection is a parenteral injection including subcutaneous injection, intradermal injection, subdermal or intramuscular injection.

Local anesthetics are usually administered by injection into the area of the nerve fibers to be blocked. Thus, absorption and distribution are not as important in controlling the onset of effect as in determining the rate of offset of anesthesia and the likelihood of central nervous system and cardiac toxicity. Topical application of local anesthetics, however, requires drug diffusion for both onset and offset of anesthetic effect. Therefore, the solubility and stability of the drug becomes major factors in determining the therapeutic effects of the drug. (Miller & Hondeghem, (1995), “Local Anesthetics” in Basic &Clinical Pharmacology, 6^{th} Edition, Ed. by Katzung).

Among the local anesthetics, lidocaine, 2-(diethylamino)-N-(2,6-dimethylphenyl)-acetamide, is particularly known for its treatment of ventricular tachycardia (an arrythmia of the heart) as an intravenous injection solution. (See e.g., U.S. Pat. No. 3,968,205). In addition, lidocaine is known for its therapeutic effects in reducing post-herpetic neuralgia (PHN) nerve injury pain from shingles (herpes zoster and post herpetic neuralgia) and analogous neuropathies. For example, U.S. Pat. No. RE37,727 discloses methods employing lidocaine intradermal administration by transport lidocaine from the skin surface, using patches and dressings, into the skin.

Formulations suitable for topical administration include liquid or semi-liquid preparations suitable for penetration through the skin to the site of where treatment is required. Examples of liquid preparations include, but are not limited to topical solution or drops (such as eye, ear, or nose drops). Examples of semi-liquid preparations include, but are not limited to liniments, lotions, creams, ointment or paste, gel, emugel. The pharmaceutical ingredients are in general those commonly used and generally recognized by person skilled in the art of pharmaceutical formulation.

### Articles of Manufacture

An article of manufacture according to the present invention includes a syringe or vial constructed of either: (i) glass; or (ii) a plastic. Examples of plastics used for syringes or vials include COP (cyclic olefin polymer) plastic, COC (cyclic olefin copolymer) plastic, high density polyethylene plastic, and high density non-nucleated polypropylene plastic.

### Buffers

The lidocaine and epinephrine containing compositions disclosed herein may further comprise a buffer (also referred to herein as buffering agent). Typically, the buffer is selected from the group consisting of phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-I-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer, and a combination thereof. Preferred buffers include phosphate buffer, bicarbonate buffer, Tris buffer, and a combination thereof. A particularly preferred buffer is phosphate buffer. A more particularly preferred buffer is sodium phosphate buffer.

Having described the many embodiments of the presently disclosed subject matter in detail, it will be apparent that modifications and variations are possible without departing from the scope of the disclosed subject matter defined in the appended claims. Furthermore, it should be appreciated that all examples in the present disclosure, while illustrating many embodiments of the disclosed subject matter, are provided as non-limiting examples and are, therefore, not to be taken as limiting the various aspects so illustrated.

Embodiments of the presently disclosed subject matter are further defined in the following examples. It should be understood that these examples are given by way of illustration only. From the above discussion and these examples, one skilled in the art can ascertain the essential characteristics of embodiments of the presently disclosed subject matter. Without departing from the spirit and scope thereof, one skilled in the art can make various changes and modifications of the disclosed subject matter to adapt it to various usages and conditions. Although specific terms have been employed herein, such terms are intended in a descriptive sense and not for purposes of limitation. All publications, including patents and non-patent literature, referred to in this specification are expressly incorporated by reference herein.

### Example: Evaluation of Duration of Anesthetic Effect and Pain on Infiltration of Different Anesthetic Solutions

### Methods

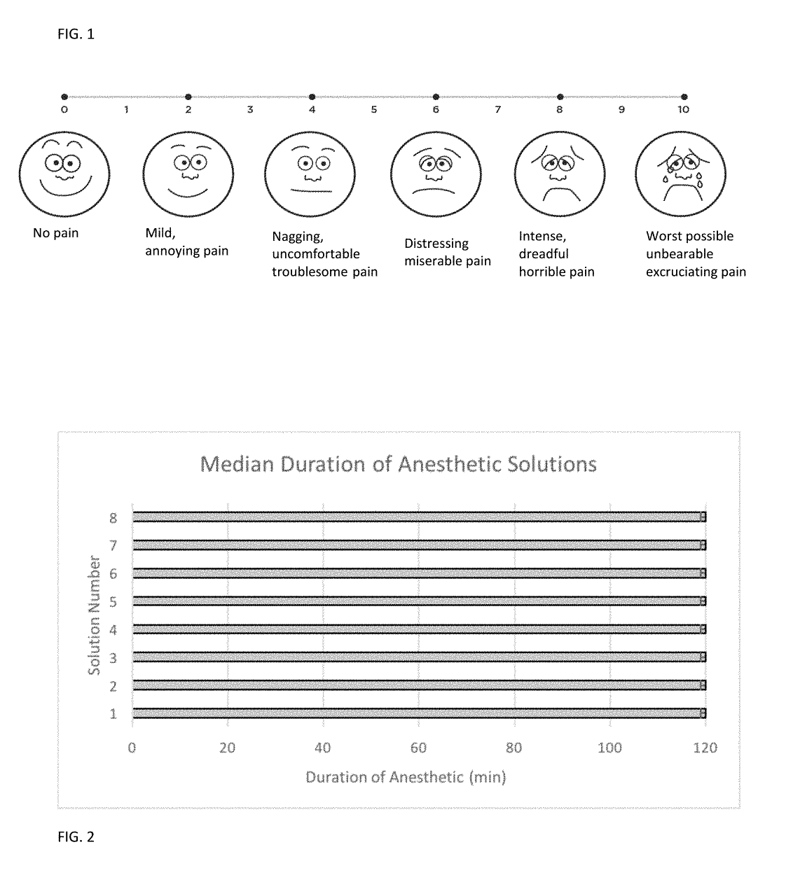

This double-blinded experiment used 30 participants to compare 7 custom solutions (solutions 2-8, described below) to a commercially available solution (solution 1) in order to directly evaluate pain on infiltration and duration of anesthesia. 0.5 ml of each local anesthetic solutions were injected into each patient intradermally. Further details of making the injectable solution are provided in Zaiac et al. J. Drugs in Dermatol, 2012; 11(10):e39-e42, incorporated herein by reference. The sites were injected in random order. Moreover, each solution was injected in a random site as well. Afterwards, the injection site was labeled with a marker to indicate which solution was injected at which site. Participants were told by the investigator to make a mental note of the maximum pain experienced during infiltration. After each injection, the participant rated their pain during infiltration using the visual analog scale (VAS) for pain (FIG. 1). This was repeated for each site of administration. Duration of anesthesia was assessed at each injection site at 15-minute intervals for a maximum of 2 hours by the presence or absence of pain to needle prick with a 30-gauge needle.

Solution 1

Lidocaine 1% w/v (lidocaine HCl), with epinephrine at a ratio of 1:100,000 (10 mg/ml Lidocaine HCL and 10 mcg/ml epinephrine, AstraZeneca)

Solution 2

Lidocaine 1% w/v with epinephrine 1:100,000 buffered 10:1 with a sodium bicarbonate 8.4% solution (Pascuet et al., Can J Hosp Pharm. 2009 September-October; 62(5): 375-380).

Solution 3

Lidocaine 0.5% w/v with epinephrine 1:200,000 (1:1 solution of lidocaine 1% with epinephrine 1:100,000 and bacteriostatic normal saline (0.9% NaCl w/v with benzyl alcohol))

Solution 4

Lidocaine 0.5% w/v with epinephrine 1:200,000 (1:1 solution of lidocaine 1% with epinephrine 1:100,000 and normal saline (0.9% NaCl w/v))

Solution 5

Lidocaine 0.25% with epinephrine 1:400,000 (1:3 solution of lidocaine 1% with epinephrine 1:100,000 with normal saline)

Solution 6

Lidocaine 0.25% with epinephrine 1:400,000 (1:3 solution of lidocaine 1% with epinephrine 1:100,000 and bacteriostatic normal saline (0.9% w/v NaCL and 0.9% w/v benzyl alcohol)).

Solution 7

Lidocaine 0.1% with epinephrine 1:1,000,000 (1:9 solution of lidocaine 1% with epinephrine 1:100,000 and bacteriostatic normal saline)

Solution 8

Lidocaine 0.1% with epinephrine 1:1,000,000 buffered (1:9 solution of lidocaine 1% and epinephrine 1:100,000 with non-bacteriostatic normal saline and buffered with sodium bicarbonate (10:1 with a sodium bicarbonate 8.4% solution))

### Discussion

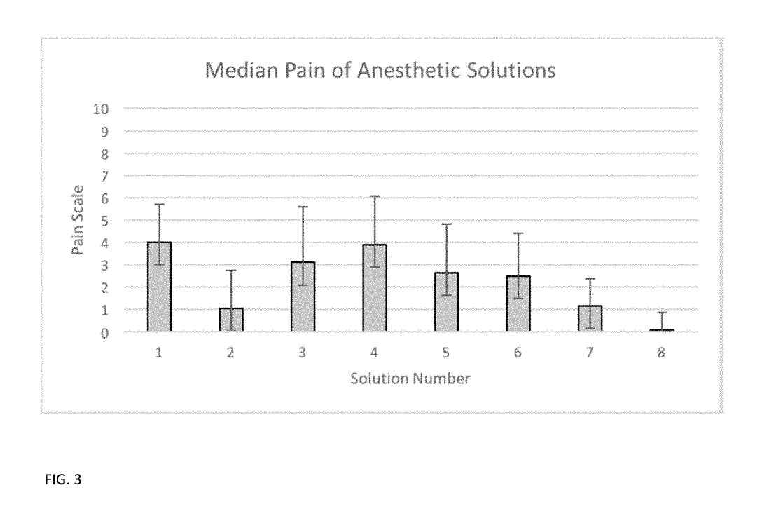

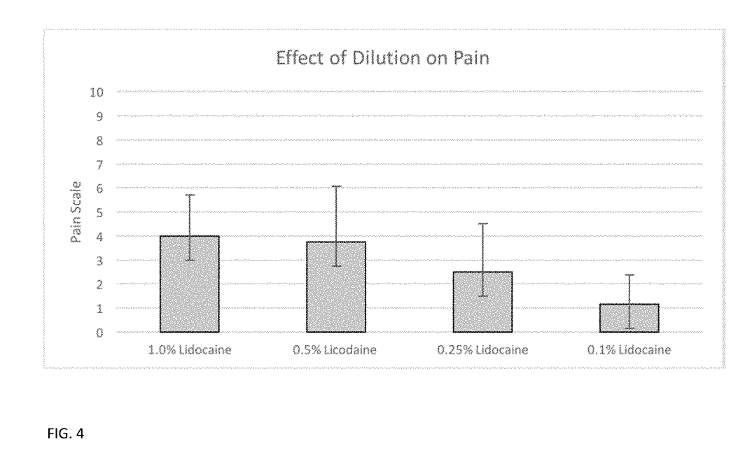

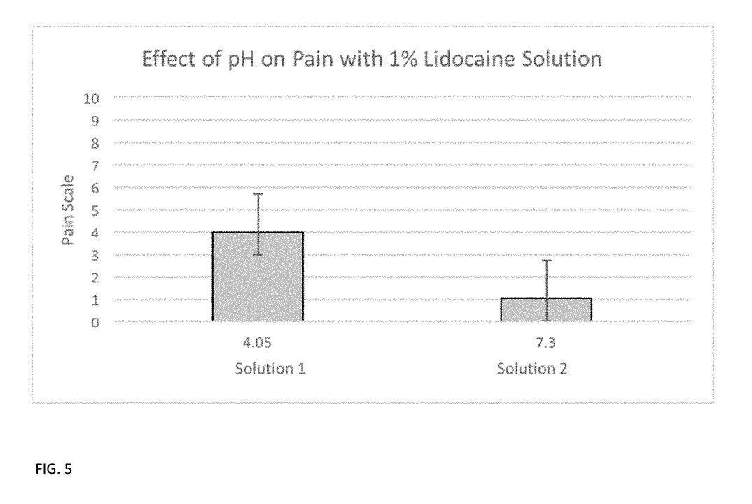

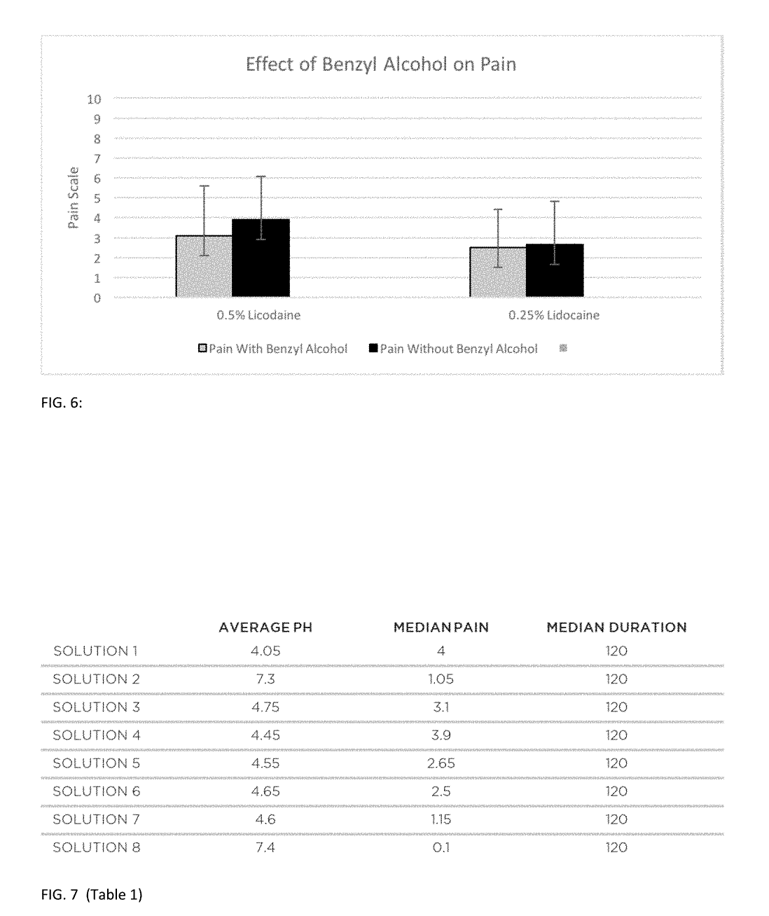

With duration of anesthetic effect proving to be equal between all solutions (FIG. 2), pain on infiltration is the key differentiator amongst these solutions (table 1, FIG. 7). Solution 1, the standard solution commonly used during cutaneous procedures, had the highest median of associated pain. It also proved to be significantly more painful than solution 2, 7, and 8 (FIG. 3). The significant difference between solution 1 and 2 can be explained by the only independent variable between the two solutions—the large difference in pH (FIG. 5, the only independent variable changed between the two solutions was pH). The significant difference between solution 1 and solutions 7 & 8 is more difficult to derive; however, other trends in our data help isolate the most likely culprits. Comparisons of solutions 3 & 4, as well as comparisons of solutions 5 & 6, show no significant difference in pain despite their difference in benzyl alcohol content, indicating that benzyl alcohol (found in bacteriostatic normal saline) likely plays no roll in mitigating pain (FIG. 6, No significant difference in pain when comparing solutions with benzyl alcohol vs. without benzyl alcohol; comparison was made at 0.5% lidocaine and at 0.25% lidocaine). Assuming that benzyl alcohol has an insignificant role in mitigating pain, the only other independent variable between solution 1 and solution 7 is the anesthetic solutions' degree of dilution (FIG. 4; Solution 7 different from solution 1 by two independent variables: concentration of lidocaine and the inclusion of benzyl alcohol (solution 7 had benzyl alcohol; solution 1 did not)). This significant difference in pain indicates that a major contributor to pain may be one of the other components in the solution: lidocaine (anesthetic), epinephrine (vasoconstrictor), methylparaben (preservative), or saline (solvent). There is some potential that the significance in pain between solution 1 and 7 could be due to the small difference in pH (solution 1=4.05; solution 7=4.60); however, that is unlikely due to the lack of significant pain difference between solution 1 and 5 (pH=4.05 and 4.55 respectively) which has an almost identical difference in pH. Lastly, solution 7 and 8 have no significant difference in pain, which—assuming benzyl alcohol has no significant effect on pain—demonstrates that at relatively dilute concentrations, pH is no longer a significant contributor to pain.

### Results

0.1% lidocaine is equally as effective as 1% lidocaine at locally anesthetizing upper extremities and other locations on the body with similar vascular supply. Decreasing the lidocaine concentration by 10 fold for standard cutaneous procedures can reduce toxicity risk and pharmacological expenses

Solution 7 had best result. It significantly decreased pain relative to the standard solution and contained the most diluted components which decreases likelihood of toxicity. It also maintained a low pH which facilitates a long shelf life

Solution 1 (standard lidocaine solution) had the highest median associated pain, and solution 8 (Klein's solution) had the lowest associated pain.

Pain on infiltration is multifactorial. Significantly decreased pain associated with relatively dilute anesthetic solution indicate pain may be caused by other constituents of the anesthetic solution such as lidocaine (anesthetic), epinephrine (vasoconstrictor), methylparaben (preservative), or saline (solvent). pH can effect pain variably depending on concentration of anesthetic solution. Benzyl alcohol has no significant impact on pain—but may be a good preservative replacement if it is found that methylparaben causes pain. Decreasing pain on infiltration via dilution, opposed to buffering, is an effective method of decreasing pain, while simultaneously maintaining shelf life.

### REFERENCES

References cited herein are incorporated herein in their entirety to the extent not inconsistent with the teachings herein.

What is claimed is:

1. A composition comprising an aqueous solution of 0.5 to 0.05% w/v lidocaine in combination with epinephrine at approximately 1:1,000,000.

2. The composition of claim 1, the lidocaine percentage is or approximately is 0.1% w/v.

3. The composition of claim 1, further comprising normal saline or bacteriostatic normal saline.

4. The composition of claim 2, further comprising normal saline or bacteriostatic normal saline.

5. The composition of claim 3, wherein the composition comprises a 1:9 solution of lidocaine (at approximately 1%) and epinephrine (at approximately 1:100,000) with bacteriostatic normal saline.

6. The composition of claim 5, wherein the comprises a 1:9 solution of 1% w/v lidocaine and epinephrine 1:100,000 with bacteriostatic normal saline.

7. The composition of claim 1, further comprising a buffering agent.

8. The composition of claim 2, further comprising a buffering agent.

9. The composition of claim 3, further comprising a buffering agent.

10. The composition of claim 7, wherein the buffering agent is sodium bicarbonate.

11. The composition of claim 8, wherein the buffering agent is sodium bicarbonate.

12. The composition of claim 9, wherein the buffering agent is sodium bicarbonate.

13. An article of manufacture comprising a container with a composition of claim 1 disposed therein.

14. The article of manufacture of claim 13, wherein the container is a vial or syringe.

15. The article of manufacture of claim 13, wherein the container comprises 10-1000 ml of the composition.

16. The article of manufacture of claim 15, wherein the container comprises 50 ml of the composition.

17. A method of inducing an anesthetic effect in a patient comprising administering an effective amount of the composition of claim 1 to the patient.

18. The method of claim 17, wherein the administering comprises intradermal injection, subcutaneous injection, subdural, or intramuscular injection of the composition.

19. A method of inducing an anesthetic effect in a patient comprising administering an effective amount of the composition of claim 5 to the patient.

20. The method of claim 19, wherein the administering comprises intradermal injection, subcutaneous injection, subdural, or intramuscular injection of the composition.
